SEQUENCE LISTING (2 sequences)

SEQ ID NO: 1 - protein, 15 aa, Artificial sequence
  synthetic AMARA peptide
AMARAASAAALARRR

SEQ ID NO: 2 - protein, 15 aa, Artificial sequence
  synthetic SAMS peptide
HMRSAMSGLHLVKRR

(19) United States
(12) Patent Application Publication
(10) Pub. No.: US 2010/0323979 A1
(43) Pub. Date: Dec. 23, 2010

(54) METHODS AND COMPOSITIONS FOR INHIBITING PROLIFERATION OF ANEUPLOID CELLS

(76) Inventors: Angelika B. Weis-Amon, Andover, MA (US); Yun-Chi Tang, Cambridge, MA (US)

Correspondence Address: WOLF GREENFIELD & SACKS, P.C., 600 ATLANTIC AVENUE, BOSTON, MA, 02210-2206, US

(21) Appl. No.: 12/817,148

(22) Filed: Jun. 16, 2010

Related U.S. Application Data
(60) Provisional application No. 61/187,657, filed on Jun. 16, 2009.

(51) Int. Cl. A61K 31/704; C12N 5/07; A61K 31/155; A61K 31/05; A61K 31/7056; A61K 31/4164; A61K 31/56; A61K 31/505; C12Q 1/68

(52) U.S. Cl. 514/34; 435/375; 514/635; 514/733; 514/43; 514/398; 514/52; 514/171; 514/274; 435/6

(57) ABSTRACT

The present invention relates, in part, to the use of AMP kinase agonists to reduce or inhibit the proliferation of aneuploid cells. The invention is also directed to the use AMP kinase agonists to reduce or inhibit the proliferation of solid tumors characterized by aneuploidy in an individual.

### RELATED APPLICATION

This application claims the benefit under 35 U.S.C. §119(e) of U.S. provisional application Ser. No. 61/187,657, filed Jun. 16, 2009, the disclosure of which is incorporated by reference herein in its entirety.

### FIELD OF THE INVENTION

The present invention relates to cancer therapy and methods of indentifying subjects for cancer treatment.

### BACKGROUND OF THE INVENTION

Tumors (benign, pre-malignant, or malignant) and unwanted mammalian cell proliferation are a major cause of morbidity and mortality. Malignant tumors (cancer) cause about 13% of all deaths. According to the American Cancer Society, 7.6 million people died from cancer in the world during 2007.

Treatment of tumors and unwanted mammalian cell proliferation can be approached by several modes of therapy, including surgery, radiation, drug therapy (e.g., chemotherapy), or a combination of any of these treatments. Chemotherapy is an indispensable form of treatment for tumors and unwanted mammalian cell proliferation. However, many chemotherapeutic agents produce unwanted side effects. A need exists to identify novel chemotherapeutic agents.

### SUMMARY OF THE INVENTION

The invention relates, in one aspect, to the discovery that aneuploid cells exhibit increased sensitivity to AMP kinase agonists. Accordingly, one aspect of the invention involves methods for reducing or inhibiting the proliferation of aneuploid cells. In some embodiments, aneuploid cells are contacted with an effective amount of an AMP kinase agonist.

According to some aspects of the invention, methods for reducing or inhibiting the proliferation of cancer (e.g., tumor) cells in an individual are provided. In some embodiments, an individual is identified on the basis that the individual is known to have an aneuploid cancer (e.g., tumor) and administering to said individual an effective amount of AMP kinase agonist to reduce or inhibit the proliferation of the cancer (e.g., tumor) cells in the aneuploid cancer (e.g., tumor). In some embodiments, an effective amount of one or more AMP kinase agonists is administered to an individual who has cancer. In some embodiments, the individual may not have been identified as having an aneuploid cancer (e.g., the cancer was not evaluated for aneuploidy or the cancer did not show present signs of aneuploidy).

According to some aspects of the invention, methods for determining if an individual should be administered an AMP kinase agonist are provided. In some embodiments, a cancer (e.g., tumor) sample is obtained from an individual and a karyotype analysis on the sample is performed to determine if the cancer (e.g., tumor) contains cells that are aneuploid. The presence of aneuploid cells in the cancer (e.g., tumor) sample indicates that an AMP kinase agonist should be administered to the individual. In some embodiments, an AMP kinase agonist is further administered to the individual. The sample may be obtained from the individual by performing a biopsy. In some embodiments, the sample may be a DNA sample or a cellular sample. According to some aspects of the invention, methods of reducing or inhibiting cancer (e.g., tumor) cells lacking a functional endogeneous tumor suppressor gene (e.g., with a mutation or deletion of one or both alleles) are provided. In some embodiments, cancer (e.g., tumor) cells lacking a functional endogeneous tumor suppressor gene are contacted with an effective amount of an AMP kinase inhibitor. In some embodiments, the tumor suppressor gene is p53. In some embodiments, the individual is known to have one or more mutations in one or more oncogenes, such as ras, c-myc, erB-2, src, and bcl-2. In some embodiments, the individual is at risk of developing cancer or has been previously diagnosed with cancer. In some embodiments, the individual has one or more other indicia or risk factors for a disease or condition associated with aneuploidy.

The following embodiments apply equally to the various aspects of the invention set forth herein unless indicated otherwise.

In some embodiments, the AMP kinase agonist is administered in combination with a therapeutic agent. In some embodiments, the therapeutic agent can target proliferating tumor cells or other proliferating cells (e.g., the therapeutic agent is cytotoxic or cytostatic to proliferating tumor cells or other proliferating cells). Examples of therapeutic agents include, but are not limited to, 5-fluorouracil (5-FU), daunomycin, doxorubicin, mitomycin, paclitaxel, rapamycin, (sirolimus), dexamethasone, 2-methoxyestradiol, and Piritrexim isethionate. However, other chemotherapeutic or other anti-tumor or anti-cancer therapeutic agents may be used as aspects of the invention are not limited in this respect.

Examples of AMP kinase agonists include, but are not limited to, Metformin (N,N-dimethylimidodicarbonimidic diamide), Resveratrol (trans-3,5,4′-Trihydroxystilbene), AICA-riboside (5-aminoimidazole-4-carboxamide riboside), AICA base (5-aminoimidazole-4-carboxamide), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues, and derivatives, including salts, of any of said compounds.

In some embodiments, the tumor is non-pancreatic. In some embodiments, the AMP kinase agonist is not Metformin or AICA-riboside.

It should be appreciated that embodiments of the methods and compositions described herein may be useful to prevent, delay, or reduce, the growth and/or proliferation of aneuploid cells in vitro and/or in vivo.

In some embodiments, aspects of the invention may be useful to treat or assist in the treatment of a disease or condition associated with aneuploidy (e.g., a tumor or cancer).

Each of the embodiments and aspects of the invention can be practised independently or combined. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the inventions, as well as various advantages and utilities will be apparent with reference to the Detailed Description. Each aspect of the invention can encompass various embodiments as will be understood.

All documents identified in this application are incorporated in their entirety herein by reference.

### BRIEF DESCRIPTION OF THE DRAWINGS

FIG. 1. AICA-riboside treatment selectively inhibits proliferation of trisomic primary MEF cells.

FIG. 2. AICAR treatment selectively inhibits proliferation of aneuploid lymphoma cells via AMPK.

FIG. 2A. Eμ-Myc;Arf−/− and Eμ-Myc;p53−/− lymphoma cells were treated with various concentrations of AICAR for 24 h. Cells were counted and expressed relative to control.

FIG. 2B. Eμ-Myc;Arf−/− and Eμ-Myc;p53−/− cells were infected with AMPK-specific shRNA and the knock down efficiencies are shown in FIG. 2C. These cells were treated with AICAR as in FIG. 2A.

FIG. 3. The proteotoxic compounds 17-AAG and chloroquine exaggerate the anti-proliferative effects of AICAR.

FIG. 3A and FIG. 3B. Wild-type (filled bars) and trisomic cells (open bars) were treated with the indicated concentrations of 17-AAG (FIG. 3A) or Chloroquine (FIG. 3B) and cell number was determined after 3 days.

FIG. 3C and FIG. 3D. Cells were treated with 0.2 mM AICAR and the indicated concentration of 17-AAG (FIG. 3C) or Chloroquine (FIG. 3D). Cell number was determined after 3 days. The data presented are the mean±standard deviation. *P<0.05, **P<0.005, t test.

FIG. 4. AICAR exaggerates the stressed state of trisomic MEFs.

FIG. 4A. Lipidated LC3-II was analyzed by immunoblotting in wild-type and trisomy 13 and 16 cells after 24 hours of AICAR treatment.

FIG. 4B. Quantitative RT-PCR analysis of mRNA abundance of the autophagy genes ATG1, ATG4, Beclin, LC3, BNIP3 and GAPRAPL1. mRNA levels were quantified in untreated wild-type (black bars) and trisomic (white bars) cells as well as AICAR treated wild-type (grey bars) and trisomic (grey bars) cells. RNA levels were normalized to those of the ribosomal rp119 gene.

FIG. 4C. The extent of autophagy was quantified by determining the number of LC3-GFP puncta in cells. Incubation in HBSS induces acute starvation and served as a positive control. 24 hours after AICAR treatment, the number of cells that harbor more than 4 LC3-GFP puncta was determined.

FIG. 4D. Wild-type and trisomic MEFs were treated with AICAR at the indicated doses and levels of inducible Hsp70 were determined by immunoblotting. The data presented are the mean±standard deviation. *P<0.05, **P<0.005, t test.

### DETAILED DESCRIPTION OF THE INVENTION

Aspects of the present invention are based, at least in part, on the finding that aneuploid cells exhibit increased sensitivity to AMP kinase agonists. Accordingly, described herein are methods for inhibiting the undesired proliferation of aneuploid cells comprising administering an effective amount of an AMP kinase agonist. Aneuploidy refers to a situation in which the chromosome content within a given cell is abnormal. The term “aneuploidy” is intended to mean a condition wherein a cell contains an abnormal number of chromosomes or segments thereof. An abnormal number of chromosomes is a number greater than or less than the normal diploid number. Whole chromosomes or segments of chromosomes may either be absent, or in addition to the normal euploid complement of chromosomes. The term also encompasses the condition wherein individual genes are present in abnormal quantity, or wherein fragments of individual genes are present in abnormal quantity. Accordingly, in humans, aneuploidy may be any deviation from the normal human diploid number of 46 chromosomes (including, for example, deletion or duplication of one or more chromosomal regions).

Aneuploidy is the leading cause of miscarriages and mental retardation in humans. Moreover, in humans more than 90% of all solid tumors are aneuploid. It should be appreciated that in the context of tumors, aneuploidy arises sporadically and can be the result of one or more mutations in cancer associated genes. Thus, compounds that selectively inhibit proliferation of aneuploid cells or kill them but do not affect normal euploid cells would be greatly effective in treating human cancers. The methods described herein may be used to identify individuals with aneuploid tumors and inhibit the proliferation of the tumor cells within these individuals. In some embodiments, the present invention provides methods of reducing or inhibiting the proliferation of a tumor (solid and/or non-solid) or an unwanted mammalian cell proliferation characterized by aneuploidy in an individual, comprising administering to the subject an effective amount of an AMP kinase agonist. In some embodiments, aneuploidy refers to an increase or decrease in the number of whole chromosome or segments of chromosomes. In some embodiments, aneuploidy results from a loss of tumor suppressor activity. According to some aspects of the invention, the tumor cells lack a functional tumor suppressor gene, such as p53.

Different organisms have widely varying chromosome complements and the term “aneuploidy” does not refer to a particular number of chromosome, but rather to an abnormal number of chromosomes. Aneuploidy includes an imbalance of genetic material caused by loss or gain of part of any chromosome (segmental aneuploidy). Accordingly, in some embodiments, aneuploid cells may have three copies of part of one chromosome and only one copy of part of the other chromosome. In other embodiments, aneuploid cells may contain an addition or deletion of one or more entire (whole) chromosomes. In other embodiments, aneuploid cells may contain an addition or deletion of one or more chromosomal arms or portions thereof. Accordingly, in some embodiments, aneuploid cells may be monosomic, trisomic, tetrasomic, etc., for one or more chromosomes or chromosomal regions. In some embodiments, aneuploid cells may have a loss of one or both copies of one or more chromosomes or chromosomal regions. In some embodiments, a region of about 0.01%, about 0.1%, about 1%, about 10%, about 25%, about 50%, or a higher, lower, or intermediate percentage of each of one or more chromosomes may be lost (e.g., one copy or both copies absent from a cell) or duplicated (e.g., three, four, or more copies in a cell). It should be appreciated that aneuploidy also may be associated with one or more additional chromosomal rearrangements including translocations, inversions, etc., of one or more chromosomal regions.

In some embodiments, without wishing to be bound by any theory, these genetic imbalances leads to imbalances in protein stoichiometry which cause the defects associated with aneuploid cells. Imbalances in the expression of proteins, particularly dimers or multimers wherein different subunits are expressed on different chromosomal regions, overload the protein quality control pathways leading to proteotoxic and energy stress in aneuploid cells. Aneuploid cells rely more heavily on the mechanisms that are employed to clear cells from excess protein or that shield cells from proteins that are not bound to their partners. Accordingly, in some embodiments, aneuploid cells are sensitive to conditions that interfere with the protein quality control pathways or place further stress on these cells. In some embodiments, methods for interfering with the protein quality control pathways and/or placing further stress on aneuploid cells are provided. In some embodiments, a method or composition useful for providing additional stress on an aneuploid cell (or a tissue or diseased region that consists of or contains aneuploid cells) may be useful prophylatically or therapeutically, alone or in combination with one or more additional agents, to treat or prevent the progression of a disease or condition (e.g., a tumor or cancer) associated with aneuploid cells. It should be appreciated that diseased tissues (e.g., tumor or cancer tissue) associated with aneuploid cells may consist entirely of aneuploid cells, may contain a subset of aneuploid cells (e.g., about 10%, about 20%, about 30%, about 40%, about 50%, about 60%, about 70%, about 80%, about 90%, or higher or lower percentages of aneuploid cells) as aspects of the invention are not limited in this respect. In some embodiments, the aneuploid cells may be homogeneous (all have the same genetic defects, for example the same genomic deletion, duplications, or combinations thereof). In some embodiments, the aneuploid cells may be heterogeneous (e.g., different cells or subsets of cells have different genetic defects, for example different extents of chromosomal deletions, duplications, or combinations thereof). It should be appreciated that certain diseases or conditions are associated with genomic instability leading to increasing levels of aneuploidy (e.g., larger amounts of genetic abnormalities within each aneuploid cells and/or more cells that are aneuploid) over time. Accordingly, aspects of the invention may be useful to treat subjects that have risk factors (e.g., one or more cancer-associated mutations) and/or indicia (e.g., low levels of genetic deletions and/or duplications) prior to the development of significant levels of aneuploidy (e.g., to prevent, reduce, or delay the development, growth or proliferation of aneuploid cells).

The possibility of a link between aneuploidy and tumor formation was first recognized in 1902 (Boveri 1902). Today, aneuploidy is considered to be a hallmark of cancer and a marker for poor clinical outcome (Ehlers et al. 2008). Aneuploidy is typically detected through karyotyping, a process in which a sample of cells is fixed and stained to create the typical light and dark chromosomal banding pattern and a picture of the chromosomes is analyzed. Other techniques include Fluorescence In Situ Hybridization (FISH), Quantitative Polymerase Chain Reaction (PCR) of Short Tandem Repeats, Quantitative Fluorescence PCR (QF-PCR), Quantitative Real-time PCR (RT-PCR) dosage analysis, Quantitative Mass Spectrometry of Single Nucleotide Polymorphisms, Spectral karyotype analysis (SKY), and Comparative Genomic Hybridization (CGH).

Karyotype analysis can be performed on a cancer (e.g., tumor) sample that has been obtained from an individual. Tumor tissue removed from an individual by a biopsy can be used as a tumor sample. In some embodiments, the cancer (e.g., tumor) sample is a cellular sample or a DNA sample. It will be appreciated that aspects of the invention are not limited to the methods of detecting aneuploidy and that any method which allows the determination of aneuploidy can be used. As used herein, an individual includes a mammal, such as a human, non-human primate, cow, rabbit, horse, pig, sheep, goat, dog, cat, or rodent such a rat, mouse or a rabbit. In some embodiments, the individual is a human. In some embodiments, the methods are employed to reduce or inhibit the proliferation of the tumor or the unwanted mammalian cell proliferation in an individual, such as a mammal.

Products and methods of the invention are useful for treating diseased conditions in which subset of cells in an individual are aneuploid, such as certain tumors, cancers, neurological disorders such as Alzheimer's disease, and/or unwanted mammalian proliferation of aneuploid cells. Tumors treatable by the compounds of the invention include, for example, benign and malignant solid tumors, and benign and malignant non-solid tumors. Examples of solid tumors include but are not limited to: biliary tract cancer, brain cancer (including glioblastomas and medulloblastomas), breast cancer, cervical cancer, choriocarcinoma, colon cancer, endometrial cancer, esophageal cancer, gastric cancer, intraepithelial neoplasms, including Bowen's disease and Paget's disease, liver cancer, lung cancer, lymphomas, including Hodgkin's disease and lymphocytic lymphomas, neuroblastomas, oral cancer, including squamous cell carcinoma, ovarian cancer, including those arising from epithelial cells, stromal cells, germ cells and mesenchymal cells, pancreatic cancer, prostate cancer, rectal cancer, renal cancer including adenocarcinoma and Wilms tumor, sarcomas, including leiomyosarcoma, rhabdomyosarcoma, liposarcoma, fibrosarcoma and osteosarcoma, skin cancer, including melanoma, Kaposi's sarcoma, basocellular cancer and squamous cell cancer, testicular cancer, including germinal tumors (seminomas, and non-seminomas such as teratomas and choriocarcinomas), stromal tumors and germ cell tumors, and thyroid cancer, including thyroid adenocarcinoma and medullary carcinoma. In some embodiments, the tumor is non-pancreatic.

Examples of non-solid tumors include but are not limited to hematological neoplasms. A hematologic neoplasm includes, for example, lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include Hodgkin's disease and non-Hodgkin's lymphoma. Chronic lymphoid leukemias include T cell chronic lymphoid leukemias and B cell chronic lymphoid leukemias.

Myeloid disorders include chronic myeloid disorders such as for instance, chronic myeloproliferative disorders, myelodysplastic syndrome and acute myeloid leukemia. Chronic myeloproliferative disorders include but are not limited to angiogenic myeloid metaplasia, essential thrombocythemia, chronic myelogenous leukemia, polycythemia vera, and atypical myeloproliferative disorders. Atypical myeloproliferative disorders include, for example, atypical Chronic Myelogenous Leukemia (CML), chronic neutrophilic leukemia, mast cell disease, and chronic eosinophilic leukemia.

Conditions of unwanted mammalian cell proliferation and treatable by this invention include familial adenomatous polyposis, dysplasia, hyperplasia (e.g., benign prostatic hyperplasia), fibrotic disorders, arteriosclerotic disorders, and dermatological disorders.

The present invention also provides methods of reducing or inhibiting the proliferation of cancer (e.g., tumor) cells lacking one or more functional tumor suppressor gene(s). Tumor suppressor genes are genes which, in their wild type alleles, express proteins that suppress abnormal cell proliferation. Mutations of tumor suppressor genes can lead to loss of functional tumor suppressor protein expression and consequently, abnormal cell proliferation which may be accompanied by aneuploidy. In some embodiments, loss of tumor suppressor activity leads to aneuploidy. Examples of tumor suppressor genes include, but are not limited to, the retinoblastoma susceptibility gene or RB gene, the protein 53 (p53) gene (NM_{—}000546.4; GI:187830767; also known as antigen NY-CO-13, phosphoprotein p53, transformation-related protein 53 (TRP53), tumor suppressor p53), the deleted in colon carcinoma (DCC) gene (NM_{—}005215.3; GI:260436868; also known as colorectal cancer suppressor) and the neurofibromatosis type 1(NF-1) tumor suppressor gene (NM_{—}001042492.2; GI:270132520). Some aspects of the invention involve identifying an individual with a tumor suppressor defect known to be associated with cancer and administering to the individual an effective amount of an AMP kinase inhibitor. In some embodiments, the tumor suppressor gene is p53.

Somatic cell mutations of the p53 gene are among the most common mutations found in human cancer and frequently coincide with aneuploidy. Accordingly, some aspects of the invention involve methods to reduce or inhibit the proliferation of cancers (e.g., tumors) lacking functional p53 comprising administering an effective amount of AMP kinase inhibitor. Methods to determine the p53 status of a tumor are known in the art and may involve mutational analysis by sequencing, DNA analysis, RNA analysis, and protein analysis to determine the lack of functional p53 protein and/or increased expression of mutated p53 protein as loss of p53 function is often associated with high levels of the altered p53 protein. Similarly, the status of any other tumor suppressor gene (such as DCC or NF-1) may be evaluated using methods known in the art, and individuals identified with defects may be administered an effective amount of one or more AMP kinase agonists.

In some embodiments, aspects of the present invention relate, at least in part to the discovery that aneuploid cells and cells lacking a tumor suppressor gene such as p53 exhibit increased sensitivity to AMP kinase agonists. Accordingly, some aspects of the invention involve identifying an individual that has a proliferative disorder characterized by aneuploidy (such as cancer) and administering to such an individual an effective amount of an AMP kinase agonist. In some embodiments, an actual karyotype analysis is performed to establish aneuploidy of the proliferative cells. However, it will be appreciated that the identification of an individual with a proliferative disorder characterized by aneuploidy need not require an actual determination of aneuploidy. Accordingly, some aspects of the invention provide methods for administering an AMP kinase agonist to an individual based on the knowledge obtained from medical records and/or patient history that the individual has a proliferative disorder characterized by aneuploidy and/or has a tumor suppressor gene mutation known to be associated with cancer. In some embodiments, karyotype analysis is not performed and AMP kinase agonists are administered to an individual based on the knowledge that the individual has a proliferative disorder characterized by aneuploidy and/or has a tumor suppressor gene mutation known to be associated with cancer.

The invention also relates to methods of making a medicament for use in treating a subject, e.g., for treating or preventing a proliferative disorder characterized by aneuploidy, and for treating or preventing cancers (e.g., tumors) having a tumor suppressor gene mutation known to be associated with cancer. Such medicaments can be used for prophylactic treatment of a subject at risk for or suspected of having an aneuploid tumor (e.g., for treatment of a subject prior to, during, and/or after the subject receives chemotherapy). Accordingly, one or more compounds or compositions described herein may be used for the preparation of a medicament for use in any of the methods of treatment described herein. In some embodiments, the invention provides for the use of one or more compounds or compositions of the invention for the manufacture of a medicament or pharmaceutical for treating a mammal (e.g., a human) having one or more symptoms of, or at risk for developing a proliferative disorder characterized by aneuploidy, and/or a tumor having a tumor suppressor gene mutation known to be associated with cancer. Accordingly, aspects of the invention relate to the use of one or more compounds or compositions of the invention for the preparation of a medicament for treating or preventing aneuploid tumors in a subject.

Accordingly, the invention also relates to one or more compounds or compositions of the invention for use as a medicament. The invention also relates to one or more of these compounds or compositions for use in methods of the invention, for example in methods of reducing or inhibiting the proliferation of aneuploid cells, or for reducing or inhibiting the proliferation of tumor cells lacking a functional endogenous tumor suppressor gene such as p53.

In some embodiments, without wishing to be bound by any theory, protein stoichiometry imbalance is a cause of defects associated with aneuploidy due to chromosome gains or losses. Cells must replicate and maintain the extra chromosomal material or compensate for the loss of chromosomal material, creating a higher demand on the DNA replication machinery and chromosome maintenance and segregation pathways. Imbalances in the expression of proteins, particularly dimers or multimers wherein different subunits are expressed on different chromosomal regions, overload the protein quality control pathways leading to proteotoxic and energy stress in aneuploid cells. Aneuploid cells rely more heavily on the mechanisms that are employed to clear cells from excess protein or that shield cells from proteins that are not bound to their partners. In both budding yeast and MEFs, the majority of genes located on an additional chromosome are expressed (Torres et al., 2007; Williams et al., 2008). In general, the amount of additional DNA present in the cells is correlated to the severity of the phenotype observed, albeit duplication or loss of certain regions (e.g., certain proteins) may be more sensitive. However, cancers are unstable, therefore any signs of aneuploidy (or risk of aneuploidy) can be used to identify an individual as a candidate for treatment.

In some embodiments, aneuploid cells exhibit increased sensitivity to conditions that interfere with transcription and protein synthesis or place further stress on the cells. In some embodiments, AMP kinase agonists exaggerate the proteotoxic and energy stress present in aneuploid cells leading to increased susceptibility. In some embodiments, an AMP kinase agonist increases the susceptibility of a tumor (or other tissue consisting of or comprising aneuploid cells) to treatment with one or more additional therapeutic agents. Accordingly, in some embodiments aspects of the invention relate to administering an AMP kinase agonist, a therapeutic agent (e.g., a chemotherapeutic agent or a compound described herein, for example in Table 1 or in the Examples), any other compound that increases stress on a cell that has a genomic and/or protein imbalance, or any combination thereof.

In some embodiments, the AMP kinase agonists are administered as prophylactics to an individual at risk of developing a condition associated with aneuploidy. For example, the AMP kinase agonists may be administered to individuals with conditions such as dysplasia, hyperplasia (such as benign prostatic hyperplasia), inflammatory bowel disorders, or other inflammatory conditions or other disorders that are associated with an increased risk of developing cancers (e.g., cancers associated with aneuploidy). The AMP kinase agonists may be administered to individuals that have a predisposition for developing cancers, such as breast, ovarian, colon and other cancers. Individuals at risk of developing cancer can be identified based on, but not limited to, individual's family medical history, individual's medical history, individuals known to have a mutant oncogene, and/or patients at least about 50 years of age. In some embodiments, the individual is otherwise free of indications calling for treatment with an AMP kinase agonist. An individual free of indications calling for treatment with an AMP kinase agonist is an individual who has no signs or symptoms calling for treatment with an AMP kinase agonist. Indications calling for treatment with an AMP kinase agonist are known to those of ordinary skill in the art. Examples of such indications include diabetes, polycystic ovary syndrome (PCOS), non-alcoholic fatty liver disease (NAFLD), and premature puberty. In some embodiments, the individual is non-diabetic (e.g. not identified as a diabetic and/or not being treated for diabetes). In some embodiments, the individual does not have polycystic ovary syndrome. In some embodiments, the individual does not have non-alcoholic fatty liver disease. In some embodiments, the individual does not have premature puberty.

In some embodiments, the aneuploid cells do not have any mutation in a nucleic acid selected from the group consisting of: Ubiquitin-specific protease gene, a DNA helicase gene, a subunit of the Set3Cdeacetylase complex gene or a promoter of ORF YJL213W or a human homologue thereof. In some embodiments, in addition to being aneuploid, the cell has at least one mutation in a nucleic acid selected from the group consisting of: Ubiquitin-specific protease gene, a DNA helicase gene, a subunit of the Set3Cdeacetylase complex gene or a promoter of ORF YJL213W or a human homologue thereof. These mutations may make the cells less susceptible to the effects of the AMP kinase agonists. However, the mutated cells may retain some sensitivity to the AMP kinase agonists that allows for the use of these compounds to reduce or inhibit the proliferation of these mutated cells. In some embodiments, the AMP kinase agonists may be administered at higher doses and/or for a longer duration of time in order to counteract the decreased sensitivity of these cells. Accordingly, in some embodiments, aspects of the invention relate to determining the presence or absence of a mutation in one or more of these genes, and administering an AMP kinase agonist if a mutation is present in one or more of these genes. The mutation may be determined at the nucleic acid or the protein level.

As used herein, an “AMP kinase agonist” is an adenosine monophosphate (AMP) mimetic or adenosine pro-drug or derivative thereof, characterized in that said AMP mimetic or adenosine pro-drug or derivative thereof stimulates AMP-activated protein kinase (AMPK). This enzyme plays a key role in cellular energy homeostasis and regulates several intracellular systems including the cellular uptake of glucose, the β-oxidation of fatty acids and the biogenesis of glucose transporter 4 (GLUT4) and mitochondria. AMP kinase agonists are molecules that can mimic the activating effect of AMP on said AMP kinase. In some embodiments, these agonists are structural analogues of AMP such as 5′-amino 4-imidazole carboxamide riboside (AICA-riboside). Other examples of AMP mimetics or adenosine pro-drugs or derivatives thereof for use in the present invention include AICA base (5-aminoimidazole-4-carboxamide), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), Metformin (N,N-dimethylimidodicarbonimidic diamide), and Resveratrol (trans-3,5,4′-Trihydroxystilbene). Some aspects of the invention also include di- and triphosphate derivatives of the compounds cited herein. The term “pro-drug” refers to compounds which are derivatives of a parent compound and can assist the parent compound to provide a medically desirable result. In some embodiments, the AMP kinase agonist is not Metformin and/or AICA-riboside.

However, it should be appreciated that other compounds (e.g., small molecules; nucleic acids, for example, regulatory RNA or DNA; polypeptides, for example, regulatory proteins or antibodies, other molecules, or any combination thereof) that stimulate AMP kinase expression levels and/or activity also may be used in methods of the invention. Accordingly, any compound that stimulates and/or increases the expression of AMP kinase may be used (e.g., alone, with one or more AMP kinase agonists, and/or with one or more therapeutic agents) to stress an aneuploid cell, prevent the growth or proliferation of aneuploid cells, treat or assist in the treatment of one or more conditions or diseases associated with aneuploidy (e.g., cancer) as described herein.

Methods to evaluate the ability of a given compound or procedure to activate the AMP kinase have been described and are known in the art. These include:

1. Evaluation of the ability of AMPK, a serine/threonine protein kinase, to phosphorylate in vivo or in vitro a given substrate. Typically, in these experiments the substrate is a protein or a peptide containing a sequence similar to the sequence surrounding the site phosphorylated by AMPK on its natural substrates. The AMARA peptide (AMARAASAAALARRR) (SEQ ID NO: 1) and the SAMS peptide (HMRSAMSGLHLVKRR) (SEQ ID NO: 2) are typically used as substrates in these studies. Briefly, AMPK-containing cell extracts or purified forms of the AMPK are incubated in the presence of a substrate, an adequate reaction mix and a tracer, usually a phosphorylated form of ATP (See Davies et al., S. P., Carling, D. and Hardie, D. G. (1989) Eur. J. Biochem. 186, 123-128 for an example).

2. AMPK has been found to be a heterotrimeric complex composed of a catalytic subunit (alpha) and two regulatory subunits (beta and gamma). Activation of AMPK leads to phosphorylation of the catalytic unit (the alpha subunit) on a given residue (Thr 172) that can be identified using specific antibodies reacting with the phosphorylated form of the AMPK alpha unit. Briefly, the ability of a given compound or procedure to activate the AMPK can be evaluated by its ability to induce phosphorylation of the Thr 172 residues on the AMPK catalytic subunit (See Fryer L G, Parbu-Patel and Carling, J. Biol. Chem 2002 277:25226 as a recent example).

3. Further, AMPK is known to regulate glucose uptake in several cell lines and tissues. Upon activation, AMPK induces glucose uptake that can be monitored using a radioactive ligand (glucose or glucose mimetic). Briefly, the ability of a given compound or procedure to activate the AMPK can be evaluated by its ability to increase glucose uptake in a given cell line or tissue (See Abbud W, Habinowski S, Zhang J Z, Kendrew J, Elkairi F S, Kemp B E, Witters L A, Ismail-Beigi F, Arch Biochem Biophys 2000 380:347).

Accordingly, the activity can be compared to the activity of a known AMP kinase agonist. In some embodiments, the agonist has at least 5%, at least 10%, at least 20%, at least 50%, at least 75%, at least 100% of the activity of a known AMP kinase agonist, such as Metformin or Resveratrol in one or more of these assays.

In some aspects, the AMP kinase agonists are administered in combination with other antitumor therapies. In some embodiments, the antitumor therapies include, but are not limited to, surgery, radiation, drug therapy (for example chemotherapy), or a combination of any of these treatments. In some aspects, the AMP kinase agonists are administered prior to the use of other antitumor therapies. In some aspects, the AMP kinase agonists are administered following the use of other antitumor therapies. For example, the AMP kinase agonists may be administered post surgery or following chemotherapy to inhibit the proliferation of any aneuploid cells that have not been surgically removed or killed by chemotherapy.

In some aspects, the AMP kinase agonists are administered in combination with other therapeutic agents. In some embodiments, the therapeutic agent is an antitumor agent(s) and/or an antiproliferative agent(s). Examples of antitumor agents that may be used in the invention include, but are not limited to: actimomycin D, actinomycin D, AD 32/Valrubicin, Adrenocortical suppressant, Adrenocorticosteroids/antagonists, adriamycin, AG3340, AG3433, alkylating agents such as melphalan and cyclophosphamide, Alkyl sulfonates, 5-Azacitidine, 5-azacytidine, Alfa 2b, Aminoglutethimide, Amsacrine (m-AMSA), Anthracenedione, Antiandrogens, Antibiotics, Antiestrogen, Antimetabolites, Antimitotic drugs, Asparaginase, AraC, Azacitidine, azathioprine, bacteriochlorophyll-a, Batimastat, BAY 12-9566, BB2516/Marmistat, BCH-4556, benzoporphyrin derivatives, Biological response modifiers, Bleomycin, BMS-182751/oral platinum, busulfan, Busulfan, bromodeozyuridine, 5-bromodeozyuridine, 2-CdA, Caelyx/liposomal doxorubicin, Campto/Levamisole, Camptosar/Irinotecan, Camptothecin, Carboplatin, carmustaine and poliferposan, Carmustine (BCNU), CDP 845, CDK4 and CDK2 inhibitors, Chlorambucil, chloroethylnitrosoureas cisplatin, CI-994, Cisplatin (cis-DDP), 2-chlorodeoxyadenosine, cladribine, CP-358 (774)/EGFR, CP-609 (754)/RAS oncogene inhibitor, CS-682, 9-AC, Cyclopax/oral paclitaxel, Cyclophosphamide, cytosine arabinoside, cytarabine, Cytarabine HCI, Cytokines, D2163, D4809/Dexifosamide, Dacarbazine, Dactinomycin, daunomycin, Daunorubicin HCI, DepoCyt, desmethylmisonidazole, 2′-deoxycoformycin, dexamethasone, diethylstilbestrol ethynyl estradiol, Differentiation Agents, docetaxel, 2,2′-difluorodeoxycytidine,2′-difluorodeoxycytidine, docetaxel etoposide, Doxil/liposomal doxorubicin, doxorubicin, Doxorubicin HCI, DX8951f, E7070, EO9, Edatrexate, Eniluracil/776C85/5FU enhancer, Enzymes, Epipodophylotoxins, Ergamisol/Levamisole, erythrohydroxynonyladenine (EHNA), estramustine, Estramustine phosphate sodium, Estrogens, Erthropoietin, etanidazole, Ethylenimine, Etoposide (V16-213), Evacet/liposomal doxorubicin, famesyl transferase inhibitor, Folic Acid analogs, FK 317, Floxuridine, Fludara/Fludarabine, fludarabine phosphate, fluorodeoxyuridine, 5-Fluorouracil (5-FU), Flutamide, fluoxymesterone, fragyline, Furtulon/Doxifluridine, Gallium Nitrite, gemcitabine, G-CSF, Gemzar/Gemcitabine, Glamolec, GM-CSF, hydroxyurea, hematoporphyrin derivatives, Hexamethylmelamine (HMM), HMR 1275/Flavopiridol, hormone analogs, Hormones and antagonists, HSP90 inhibitors such as 17-(Allylamino)-17-demethoxygeldanamycin (17-AAG), Hycamtin/Topotecan, hydroxyprogesterone acetate, hydroxyprogesterone caproate, Hydroxyurea (hydroxycarbamide), Idarubicin, Inhibitors, Ifes/Mesnex/Ifosamide, Ifosfamide, 5-iododeoxyuridine, Incel/VX-710, iodine seeds, interferon-alpha, interferon-β, interfon-γ, Interferon Alfa-2a, Interleukin-2, IL-2, irinotecan, IS1641, L-asparaginase, L-Buthiamine Sulfoxide,leuprolide, Lemonal DP 2202, Leuprolide acetate (LHRH-releasing factor analogue), Leustatin/Cladribine, Lomustine (CCNU), LU 79553/Bis-Naphtalimide, LU 103793/Dolastain, LY264618/Lometexol, Mechlorethamine HCI (nitrogen mustard), medroxyprogesterone acetate, Megestrol, megestrol acetate mitotane, Meglamine GLA, melphalan, 6-Mercaptopurine, Mesna, Metastron/strontium derivative, Metaret/Suramin, metronidazole, Methotrexate (MTX), Methyl glyoxal bis-guanylhydrazone (MGBG), Methylhydrazine derivatives, Methylmelamine, misonidazole, Mitoguazone (methyl-GAG), Mitomycin C, mithramycin, Mitotane (o.p′-DDD), mitoxantrone, Mitoxantrone HCI, MMI270, MMP, MTA/LY231514, naphthalocyanine, naphthalocyanines, nicotinamide, nimorazole, Npe6, Nitrogen mustards, Nitrosourceas, N-methylhydrazine, N-methylhydrazine (MIH), Nonsteroidal antiandrogens, Novantrone/Mitroxantrone, ODN 698, Octreotide, Oral Taxoid, paclitaxel, Paraplatin/Carboplatin, PARP inhibitors, Paxex/Paclitaxel, Pentostatin, PD183805, Pharmarubicin/Epirubicin, pheoboride-a, photofrin®, Photosensitizers, phthalocyanine, Picibanil/OK-432, pimonidazole, pimonidazole etanidazole, PKC412, Plantinol/cisplatin, Platinium coordination complexes, Plicamycin, poliferposan, Prednisone, prednisone and equivalents, procarbazine, Procarbazine HCI, Progestins, Purine analogs, Pyrimidine analogs, Radiosensitizers, RAS famesyl transferase inhibitor, retinoic acid derivatives, rubidomycin, RB 6145, RSU 1069, SR4233, Semustine (methyl-CCNU), Semustine Streptozocin, SPU-077/Cisplatin, Substituted urea, TA 2516/Marmistat, tamoxifen, Tamoxifen citrate, Taxane Analog, Taxanes, taxol, Taxol/Paclitaxel, Taxoids, Taxotere, Taxotere/Docetaxel, prodrug of guanine arabinoside, Temodal/Temozolomide, teniposide, Teniposide (VM-26), testosterone propionate, Thioguanine, Thiophosphoramide, 6-Thioguanine, Thiotepa, tin etioporphyrin (SnET2), Thriethylenemelamine, TNP-470, triethylene thiophosphoramide, Tiasofuran, tin etioporphyrin, Topotecan, Triazines, Triethylene, trimetrexate, Tumodex/Ralitrexed, Type I Topoisomerase, UFT(Tegafur/Uracil), valrubicin, Valspodar/PSC833, Vepeside/Etoposide, vinblastine, vinblastine (VLB), Vinblastine sulfate, Vinca alkaloids, vincristine, Vincristine sulfate, vinorelbine, VX-853, Vumon/Teniposide, ZD0101, Xeload/Capecitabine, Yewtaxan/Placlitaxel, YM 116, ZD 0473/Anormed, ZD1839, ZD 9331, or zinc phthalocyanine.

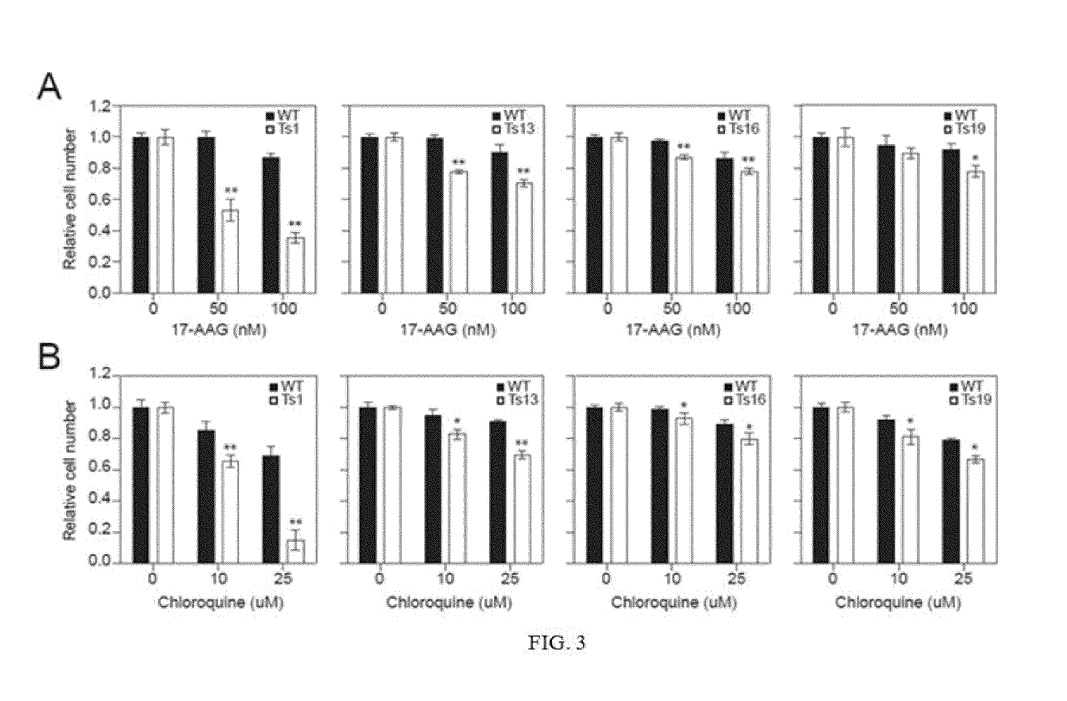

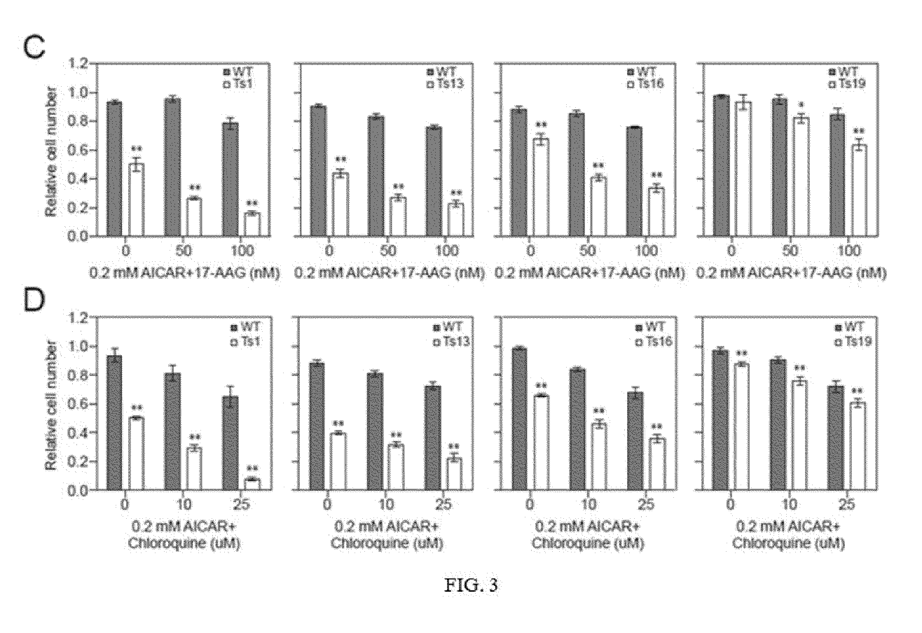

In some embodiments, the antitumor agent is 17-(Allylamino)-17-demethoxygeldanamycin (17-AAG). 17-AAG inhibits the molecular chaperone Hsp90. Hsp90 functions together with other chaperones in the folding, activation and assembly of a specific set of client proteins, including a multitude of cell-signaling proteins such as steroid hormone receptors and oncogenic protein kinases (Young et al. 2001). 17-AAG was found to preferentially inhibit the proliferation of trisomic cells (FIG. 3A).

Proliferation of trisomy 1, 13, 16 and 19 cells was impaired at a concentration of 100 nM of 17-AAG. Furthermore, cells trisomic for the largest chromosomes 1 exhibited higher sensitivity to the compound than cells harboring an additional copy of the smaller chromosomes 16 or 19. Thus, compounds that induce proteotoxic stress are detrimental for trisomic MEFs. This finding suggests that aneuploid cells rely on protein quality control pathways for their survival. Accordingly, some aspects of the invention involve administering to an individual known to have aneuploid tumors an effective amount of 17-AAG alone to reduce or inhibit the proliferation of aneuploid tumor cells. In some embodiments, 17-AAG is administered in combination with other therapeutic drugs.

In some embodiments, the therapeutic agent is chloroquine. Chloroquine induces proteotoxic stress. The drug inhibits late stages of autophagy—the fusion of autosomal vesicles with the lysosome—by raising the intra-lysosomal pH (Glaumann et al. 1987). Autophagy is a homeostatic mechanism critical for the elimination of damaged proteins and organelles (Levine et al. 2008; Mizushima et al. 2008). Under low nutrient and other stress conditions, the pathway becomes a pro-survival pathway, where cells catabolize intracellular components to ensure survival. Chloroquine was found to preferentially inhibit the proliferation of aneuploid cells. As observed for AICAR and 17-AAG, the increased sensitivity of aneuploid cells correlated with the size of the additional chromosome (FIG. 3B). Thus, interference with autophagy is detrimental in aneuploid MEFs. In some embodiment, without wishing to be bound by any theory, aneuploid cells rely on autophagy to produce energy and/or reduce proteotoxic stress. Accordingly, some aspects of the invention involve administering to an individual known to have aneuploid tumors an effective amount of chloroquine alone to reduce or inhibit the proliferation of aneuploid tumor cells. In some embodiments, chloroquine is administered in combination with other therapeutic drugs.

Compounds identified as antiproliferative in aneuploid cells were combined to determine their additive effects. The combined treatment of trisomic cells with AICAR and 17-AAG severely impaired their proliferative abilities but had little effect on euploid control cultures (FIG. 3C). Similar results were obtained when cells were treated with a combination of AICAR and chloroquine (FIG. 3D). Thus, compounds exist that selectively inhibit the proliferation of aneuploid cells. Their combined application, especially, leads to dramatic differential effects in euploid and aneuploid cells. Accordingly, in some embodiments, 17-AAG and/or chloroquine may be administered in conjunction with one or more AMP kinase agonists to an individual known to have aneuploid tumors. In some aspects, the combination of one or more AMP kinase agonists and 17-AAG and/or chloroquine may be further administered with other therapeutic agents. In some embodiments, the therapeutic agent is an antitumor agent(s) and/or an antiproliferative agent(s). Examples of other therapeutic agents include, but are not limited to those described herein.

Other examples of antitumor agents that may be used in the invention are listed in Table 1.

TABLE 1

| Marketer | Brand Name | Generic Name |
|---|---|---|
| Abbott | TNP 470/AGM 1470 | Fragyline |
| Takeda | TNP 470/AGM 1470 | Fragyline |
| Scotia | Meglamine GLA | Meglamine GLA |
| Medeva | Valstar | Valrubicin |
| Medeva | Valstar | Valrubicin |
| Rhone Poulenc | Gliadel Wafer | Carmustaine + Polifepr Osan |
| Warner Lambert | Undisclosed Cancer (b) | Undisclosed Cancer (b) |
| Bristol Myers Squib | RAS Famesyl Transferase | RAS FamesylTransferase |
| | Inhibitor | Inhibitor |
| Novartis | MMI 270 | MMI 270 |
| Bayer | BAY 12-9566 | BAY 12-9566 |
| Merck | Famesyl Transferase Inhibitor | Famesyl Transferase Inhibitor |
| Pfizer | PFE | MMP |
| Pfizer | PFE | Tyrosine Kinase |
| Lilly | MTA/LY 231514 | MTA/LY 231514 |
| Lilly | LY 264618/Lometexol | Lometexol |
| Scotia | Glamolec | LiGLA (lithium-gamma linolenate) |
| Warner Lambert | CI-994 | CI-994 |
| Schering AG | Angiogenesis inhibitor | Angiogenesis Inhibitor |
| Takeda | TNP-470 | n/k |
| Smithkline Beecham | Hycamtin | Topotecan |
| Novartis | PKC 412 | PKC 412 |
| Novartis | Valspodar | PSC 833 |
| Immunex | Novantrone | Mitoxantrone |
| Warner Lambert | Metaret | Suramin |
| Genentech | Anti-VEGF | Anti-VEGF |
| British Biotech | Batimastat | Batimastat (BB94) |
| Eisai | E 7070 | E 7070 |
| Biochem Pharma | BCH-4556 | BCH-4556 |
| Sankyo | CS-682 | CS-682 |
| Agouron | AG2037 | AG2037 |
| IDEC Pharma | 9-AC | 9-AC |
| Agouron | VEGF/b-FGF Inhibitors | VEGF/b-FGF Inhibitors |
| Agouron | AG3340 | AG3340 |
| Vertex | Incel | VX-710 |
| Vertex | VX-853 | VX-853 |
| Zeneca | ZD 0101 (inj) | ZD 0101 |
| Novartis | ISI 641 | ISI 641 |
| Novartis | ODN 698 | ODN 698 |
| Tanube Seiyaku | TA 2516 | Marimistat |
| British Biotech | Marimastat | Marimastat (BB 2516) |
| Celltech | CDP 845 | Aggrecanase Inhibitor |
| Chiroscience | D2163 | D2163 |
| Warner Lambert | PD 183805 | PD 183805 |
| Daiichi | DX8951f | DX8951f |
| Daiichi | Lemonal DP 2202 | Lemonal DP 2202 |
| Fujisawa | FK 317 | FK 317 |
| Chugai | Picibanil | OK-432 |
| Nycomed Amersham | AD 32/valrubicin | Valrubicin |
| Nycomed Amersham | Metastron | Strontium Derivative |
| Schering Plough | Temodal | Temozolomide |
| Schering Plough | Temodal | Temozolonide |
| Liposome | Evacet | Doxorubicin, Liposomal |
| Nycomed Amersham | Yewtaxan | Paclitaxel |
| Bristol Myers Squib | Taxol | Paclitaxel |
| Roche | Xeloda | Capecitabine |
| Roche | Furtulon | Doxifluridine |
| Pharmacia & Upjohn | Adriamycin | Doxorubicin |
| Ivax | Cyclopax | Paclitaxel, Oral |
| Rhone Poulenc | Oral Taxoid | Oral Taxoid |
| AHP | Novantrone | Mitoxantrone |
| Sequus | SPI-077 | Cisplatin, Stealth |
| Hoechst | HMR 1275 | Flavopiridol |
| Pfizer | CP-358, 774 | EGFR |
| Pfizer | CP-609, 754 | RAS Oncogene Inhibitor |
| Bristol Myers Squib | BMS-182751 | Oral Platinum |
| Bristol Myers Squib | UFT (Tegafur/Uracil) | UFT (Tegafur/Uracil) |
| Johnson & Johnson | Ergamisol | Levamisole |
| Glaxo Wellcome | Eniluracil/776C85 | 5FU Enhancer |
| Johnson & Johnson | Ergamisol | Levamisole |
| Rhone Poulenc | Campto | Irinotecan |
| Pharmacia & Upjohn | Camptosar | Irinotecan |
| Zeneca | Tomudex | Ralitrexed |
| Johnson & Johnson | Leustain | Cladribine |
| Ivax | Paxene | Paclitaxel |
| Sequus | Doxil | Doxorubicin, Liposomal |
| Sequus | Caelyx | Doxorubicin, Liposomal |
| Schering AG | Fludara | Fludarabine |
| Pharmacia & Upjohn | Pharmorubicin | Epirubicin |
| Chiron | DepoCyt | DepoCyt |
| Zeneca | ZD1839 | ZD 1839 |
| BASF | LU 79553 | Bis-Naphtalimide |
| BASF | LU 103793 | Dolastain |
| Shering Plough | Caetyx | Doxorubicin-Liposome |
| Lilly | Gemzar | Gemcitabine |
| Zeneca | ZD 0473/Anormed | ZD 0473/Anormed |
| Yamanouchi | YM 116 | YM 116 |
| Nycomed Amersham | Seeds/I-125 Rapid St | Lodine Seeds |
| Agouron | Cdk4/cdk2 inhibitors | cdk4/cdk2 inhibitors |
| Agouron | PARP inhibitors | PARP Inhibitors |
| Chiroscience | D4809 | Dexifosamide |
| Bristol Myers Squib | UFT (Tegafur/Uracil) | UFT (Tegafur/Uracil) |
| Sankyo | Krestin | Krestin |
| Asta Medica | Ifex/Mesnex | Ifosamide |
| Bristol Meyers Squib | Ifex/Mesnex | Ifosamide |
| Bristol Myers Squib | Vumon | Teniposide |
| Bristol Myers Squib | Paraplatin | Carboplatin |
| Bristol Myers Squib | Plantinol | Cisplatin, Stealth |
| Bristol Myers Squib | Plantinol | Cisplatin |
| Bristol Myers Squib | Vepeside | Etoposide |
| Zeneca | ZD 9331 | ZD 9331 |
| Chugai | Taxotere | Docetaxel |
| Rhone Poulenc | Taxotere | Docetaxel |
| Glaxo Wellcome | Prodrug of guanine | Prodrug of arabinside |
| | arabinside | |
| Bristol Myers Squib | Taxane Analog | Taxane Analog |

Accordingly, one of more of these therapeutic agents may be used alone or in combination with one or more AMP kinase agonists according to the methods described herein. The AMP kinase agonists may be administered alone or in conjunction with one or more therapies known or believed to be useful for treating a tumor. Such therapies include, for example, tumor medicaments, radiation and surgical procedures. As used herein, a tumor medicament refers to an agent which is administered to a subject for the purpose of treating a tumor. Various types of medicaments for the treatment of tumors are described herein. For the purpose of this specification, tumor medicaments are classified as chemotherapeutic agents, immunotherapeutic agents, tumor vaccines, hormone therapy, and biological response modifiers.

Tumor medicaments function in a variety of ways. Some tumor medicaments work by targeting physiological mechanisms that are specific to tumor cells. Examples include the targeting of specific genes and their gene products (e.g., proteins primarily) which are mutated in tumors. Such genes include but are not limited to oncogenes (e.g., Ras, Her2, bcl-2), tumor suppressor genes (e.g., EGF, p53, Rb), and cell cycle targets (e.g., CDK4, p21, telomerase). Tumor medicaments can alternately target signal transduction pathways and molecular mechanisms which are altered in tumor cells. Targeting of tumor cells via the epitopes expressed on their cell surface is accomplished through the use of monoclonal antibodies. This latter type of tumor medicament is generally referred to herein as immunotherapy.

Other tumor medicaments target cells other than tumor cells. For example, some medicaments prime the immune system to attack tumor cells (e.g., tumor vaccines). Still other medicaments, called angiogenesis inhibitors, function by attacking the blood supply of tumors. Since most malignant tumors are able to metastasize (e.g., exit the primary tumor site and seed a distal tissue, thereby forming a secondary tumor), medicaments that impede this metastasis are also useful in the treatment of tumor. Angiogenic mediators include basic FGF, VEGF, angiopoietins, angiostatin, endostatin, TNF-α, TNP-470, thrombospondin-1, platelet factor 4, CAI, and certain members of the integrin family of proteins. One category of this type of medicament is a metalloproteinase inhibitor, which inhibits the enzymes used by the tumor cells to exit the primary tumor site and extravasate into another tissue.

The AMP kinase agonist(s) may be combined with immunotherapeutic agents which are medicaments that derive from antibodies or antibody fragments which specifically bind or recognize a tumor antigen. As used herein a tumor antigen is broadly defined as an antigen expressed by a tumor cell. In some embodiments, the antigen is expressed at the cell surface of the tumor cell. In other embodiments, the antigen is one which is not expressed by normal cells, or at least not expressed to the same level as in tumor cells. Antibody-based immunotherapies may function by binding to the cell surface of a tumor cell and thereby stimulate the endogenous immune system to attack the tumor cell. Another way in which antibody-based therapy functions is as a delivery system for the specific targeting of toxic substances to tumor cells. Antibodies are usually conjugated to toxins such as ricin (e.g., from castor beans), calicheamicin and maytansinoids, to radioactive isotopes such as Iodine-131 and Yttrium-90, to chemotherapeutic agents (as described herein), or to biological response modifiers. In this way, the toxic substances can be concentrated in the region of the tumor and non-specific toxicity to normal cells can be minimized. In addition to the use of antibodies which are specific for tumor antigens, antibodies which bind to vasculature, such as those which bind to endothelial cells, are also useful in the invention. This is because generally solid tumors are dependent upon newly formed blood vessels to survive, and thus most tumors are capable of recruiting and stimulating the growth of new blood vessels. As a result, one strategy of many tumor medicaments is to attack the blood vessels feeding a tumor and/or the connective tissues (or stroma) supporting such blood vessels.

Other types of chemotherapeutic agents which can be used according to the invention in combination with AMP kinase agonist(s) include Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erythropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate.

Tumor vaccines are medicaments which are intended to stimulate an endogenous immune response against tumor cells. Currently produced vaccines predominantly activate the humoral immune system (e.g., the antibody dependent immune response). Other vaccines currently in development are focused on activating the cell-mediated immune system including cytotoxic T lymphocytes which are capable of killing tumor cells. Tumor vaccines generally enhance the presentation of tumor antigens to both antigen presenting cells (e.g., macrophages and dendritic cells) and/or to other immune cells such as T cells, B cells, and NK cells. In some instances, tumor vaccines may be used along with adjuvants, such as those described above.

Tumor antigens, such as those present in tumor vaccines or those used to prepare tumor immunotherapies, can be prepared from crude tumor cell extracts, as described in Cohen P A et al. (1994) Cancer Res 54:1055-8, or by partially purifying the antigens, using recombinant technology, or de novo synthesis of known antigens. Tumor antigens can be used in combination with AMP kinase agonist(s) in the form of immunogenic portions of a particular antigen or in some instances a whole cell or a tumor mass can be used as the antigen. Such antigens can be isolated or prepared recombinantly or by any other means known in the art.

Other vaccines take the form of dendritic cells which have been exposed to tumor antigens in vitro, have processed the antigens and are able to express the tumor antigens at their cell surface in the context of MHC molecules for effective antigen presentation to other immune system cells. Dendritic cells form the link between the innate and the acquired immune system by presenting antigens and through their expression of pattern recognition receptors which detect microbial molecules like LPS in their local environment.

Examples of antiproliferative agents that may be used in the invention in conjunction with AMP kinase agonist(s) include, but are not limited to: 5-fluorouracil (5-FU), daunomycin, doxorubicin, mitomycin, paclitaxel, rapamycin, (sirolimus), dexamethasone, 2-methoxyestradiol, and Piritrexim isethionate.

The AMP kinase agonist(s) and other therapeutic agent(s) may be administered simultaneously or sequentially. When the other therapeutic agents are administered simultaneously they can be administered in the same or separate formulations, but are administered at the same time. The administration of the other therapeutic agents and the AMP kinase agonist(s) may also be temporally separated, meaning that the therapeutic agents are administered at a different time, either before or after, the administration of the AMP kinase agonist(s). The separation in time between the administration of these compounds may be a matter of minutes or it may be longer.

The compounds of the invention are administered in effective amounts. An effective amount is a dosage of the therapeutic compounds sufficient to provide a medically desirable result and can be determined by one of skill in the art using routine methods. An effective amount means that amount necessary to delay the onset of, inhibit the progression of or halt altogether the onset or progression of the particular condition or disease being treated. In the treatment of tumors or unwanted mammalian cell proliferation, for example, in general, an effective amount will be that amount necessary to inhibit tumor growth or progression of the tumor or the unwanted mammalian cell proliferation, reduce the load of the tumor or the unwanted mammalian cell proliferation, or reduce one or more signs or symptoms of the tumor or unwanted mammalian cell proliferation. In some embodiments, an effective amount is an amount which results in any improvement in the particular condition or disease being treated. In some embodiments, an effective amount is an amount that interferes with the protein quality control pathways and/or places further stress on aneuploid cells. In some embodiments, an effective amount may depend on the type of cancer, the extent of aneuploidy, and/or the use of one or more additional therapeutic agents. However, one of skill in the art can determine appropriate doses and ranges of AMP kinase agonists to use, for example based on in vitro and/or in vivo testing and/or other knowledge of compound dosages. It should be appreciated that in some embodiments, one or more compositions described herein may be cytostatic or cytotoxic for aneuploid cells (e.g., proliferating aneuploid cells). Accordingly, in some embodiments, methods and compositions of the invention may be administered in dosages that prevent the growth or proliferation of aneuploid cells, and/or kill aneuploid cells, without interfering or killing healthy cells and/or without preventing normal growth, development, and/or replication of healthy (e.g., non-aneuploid cells).

When administered to a subject, effective amounts of AMP kinase agonist(s) will depend, of course, on the particular condition being treated; the severity of the condition; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

An effective amount typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 500 mg to 1000 mg per day.

Actual dosage levels of active ingredients in the pharmaceutical compositions of the invention can be varied to obtain an amount of the active compound(s) that is effective to achieve the desired therapeutic response for a particular patient, compositions, and mode of administration. The selected dosage level depends upon the activity of the particular compound, the route of administration, the severity of the condition being treated, the condition, and prior medical history of the patient being treated. However, it is within the skill of the art to start doses of the compound at levels lower than required to achieve the desired therapeutic effort and to gradually increase the dosage until the desired effect is achieved.

The dosage regimen can be determined, for example, by following the response to the treatment in terms clinical signs. Examples of such clinical signs are well known in the art, and they include for example the pulse, blood pressure, temperature, and respiratory rate. Harrison's Principles of Internal Medicine, 15th Ed., Fauci A S et al., eds., McGraw-Hill, New York, 2001.

The compounds and pharmaceutical compositions of the invention can be administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, brain, or in the cornea. In some embodiments, the compositions may be contacted or administered directly to the aneuploid cells. In some embodiments, the compositions may be administered systemically.

The AMP kinase agonist(s) also can be administered in the form of liposomes. As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. In some embodiments, the lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33, et seq.

Dosage forms for topical administration of a compound of this invention include powders, sprays, ointments, and inhalants as described herein. The active compound is mixed under sterile conditions with a pharmaceutically acceptable carrier and any needed preservatives, buffers, or propellants which may be required. Ophthalmic formulations, eye ointments, powders, and solutions also are contemplated as being within the scope of this invention.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the drug, it is desirable to slow the absorption of the drug from subcutaneous or intramuscular injection. This result can be accomplished by the use of a liquid suspension of crystalline or amorphous materials with poor water solubility. The rate of absorption of the drug then depends upon its rate of dissolution which, in turn, may depend upon crystal size and crystalline form. Alternatively, delayed absorption of a parenterally administered drug from is accomplished by dissolving or suspending the drug in an oil vehicle.

Injectable depot forms are made by forming microencapsule matrices of the drug in biodegradable polymers such a polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation includes a compound of the invention and inert ingredients which protect against degradation in the stomach and which permit release of the biologically active material in the intestine.

In such solid dosage forms, the active compound is mixed with, or chemically modified to include, at least one inert, pharmaceutically acceptable excipient or carrier. The excipient or carrier may permit (a) inhibition of proteolysis, and (b) uptake into the blood stream from the stomach or intestine. In some embodiments, the excipient or carrier increases uptake of the compound, overall stability of the compound and/or circulation time of the compound in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropyhnethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffm, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions which can be used include polymeric substances and waxes.

The active compounds also can be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the active compounds, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Also contemplated herein is pulmonary delivery of the compounds of the invention. The compound is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (suppl.5): s.143-146 (1989)(endothelin-1); Hubbard et al., Annals of Internal Medicine 3:206-212 (1989) (α1-antitrypsin); Smith et al., J. Clin. Invest. 84:1145-1146 (1989) (cd-proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a compound of the invention. Typically, each formulation is specific to the type of device employed and can involve the use of an appropriate propellant material, in addition to diluents, adjuvants, and/or carriers useful in therapy.

The composition may be prepared in particulate form, with an average particle size of less than 10 μm, or 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

Also, the use of liposomes, microcapsules or microspheres, inclusion complexes, or other types of carriers is contemplated.

Formulations suitable for use with a nebulizer, either jet or ultrasonic, typically comprise a compound of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the inhibitor compound suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the inhibitor and also can include a bulking agent, such as lactose, sorbitol, sucrose, mannitol, trehalose, or xylitol, in amounts which facilitate dispersal of the powder from the device, e.g., 50 to 90% by weight of the formulation.

Nasal delivery of the compounds and composition of the invention also is contemplated. Nasal delivery allows the passage of the compound or composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

In some embodiments, compositions for rectal or vaginal administration are suppositories which can be prepared by mixing the compounds of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compound.

In order to facilitate delivery of compounds across cell and/or nuclear membranes, compositions of relatively high hybrophobicity are preferred. Compounds can be modified in a manner which increases hydrophobicity, or the compounds can be encapsulated in hydrophobic carriers or solutions which result in increased hydrophobicity.

Generally dosage levels of about 0.1 to about 1000 mg, about 0.5 to about 500 mg, about 1 to about 250 mg, about 1.5 to about 100, and of about 5 to about 20 mg of active compound per kilogram of body weight per day are administered orally or intravenously. If desired, the effective daily dose can be divided into multiple doses for purposes of administration, e.g., two to four separate doses per day.

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

### Example

### Materials and Methods

### Cell Lines and Tissue Culture

All euploid (WT) and aneuploid (Ts) mouse embryonic fibroblast (MEF) lines were established as previously described (Williams, B. R. et al., 2008). MEF cells were cultured in DMEM supplemented with 10% FBS, 2 mM glutamine, 50 U/ml penicillin, and 50 U/ml streptomycin. Eμ-Myc;Arf−/− and Eμ-Myc;p53−/− lymphoma cells were established as previously described (Wendel, H. et al., 2006). Lymphoma cells were cultured in IMDM supplemented with 10% FBS, 2 mM glutamine, 50U/ml penicillin, 50 U/ml streptomycin and 50 mM beta-mercaptoethanol. All cells were cultured at 37° C. with 5% CO₂ in a humidified environment. In all experiments, cells were counted using a Cellometer Auto T4 automated hemacytometer (Nexcelom).

### Short Hairpin RNA Vectors

AMPK-specific shRNA constructs were generated as described previously (Silva, J. M. et al., 2005). Targeting sequences were selected based on BIOPREDsi design (Huesken, D, et al., 2005).

### Cell Proliferation Assay

Exponentially growing early passage MEFs were plated at a density of 2×10⁵ cells/well in 6-well plates and lymphoma cells were cultured and infected as described previously (Hemann, M. T. et al., 2003). After infection with shRNA constructs, GFP+FACS sorted cells were plated at a density of 5×10⁵ cells/well in 6-well plates. Cells were treated with AICAR at various concentrations or solvent control then counted at indicated time point.

### Western Blot Analysis

Western blotting was performed as previously described (Dickins, R. A. et al., 2005). Proteins were detected by using the following antibodies: anti-AMPKα (Cell signaling technology), anti-Hsp70 (Stressgen), and anti-β actin (Sigma). Secondary antibodies were horseradish peroxidase-conjugated anti-mouse or anti-rabbit IgG (GE Healthcare; 1:5,000).

### Fluorescence Imaging

For GFP-LC3 fluorescence imaging, wild-type GFP-LC3 and trisomic GFP-LC3 MEF cells were exposed to the indicated drug treatments in phenol red-free DMEM. All fluorescence images were acquired using a Nikon digital sight DS-Qil MC camera on a Nikon Eclipse Ti inverted microscope. All single plane images were acquired and processed using the NIS-Element AR 3.0 software.

### RT Quantitative PCR

Total RNA from MEFs was isolated using the RNeasy Mini Kit (Qiagen) according to the manufacturer's instructions. 1μg RNA was used to generate cDNAs using RETROscript kit (Ambion). qPCR was performed by mixing iQ SYBR Green Supermix (Bio-Rad) with the primers described in Table S2, and amplified using a LightCycler 480 II (Roche). The LightCycler software was used to evaluate gene expression level and normalized to a ribosomal reference gene, rpl19.

### Results

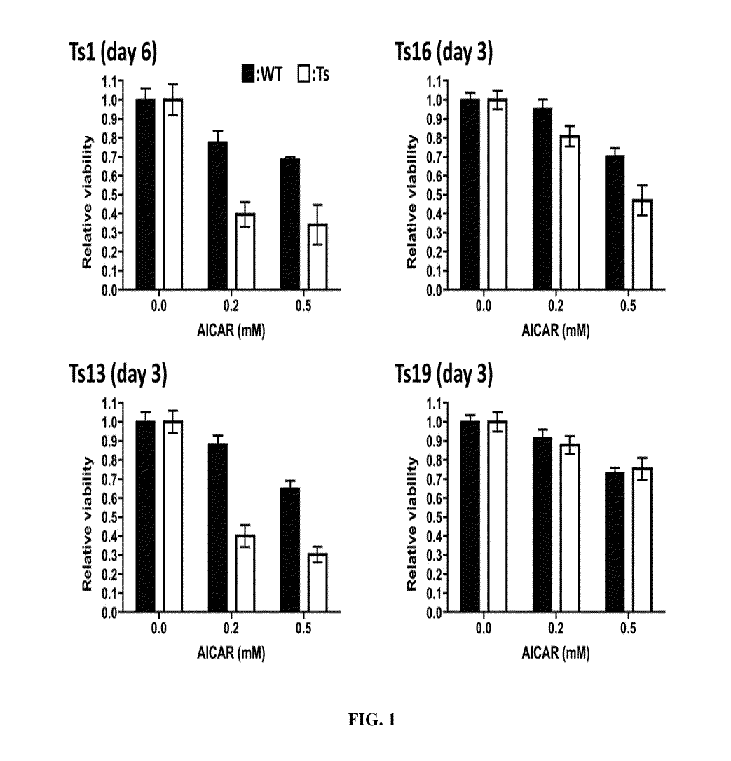

FIG. 1 demonstrates that mouse embryonic fibroblasts (MEFs) carrying different trisomies (Trisomic for chromosome 1 or 13 or 16 or 19) are sensitive to AICA-riboside. All cell lines were treated for 72 or 144 h with various concentrations of AICA-riboside (0-0.5 mM), and cells were counted at indicated time points. The results are expressed relative to non-treated control.

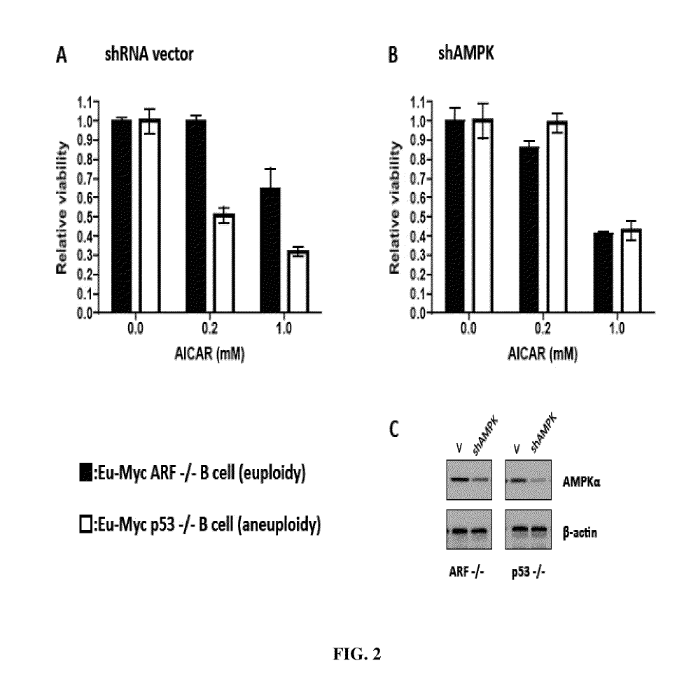

FIG. 2A shows that euploid (meaning correct chromosome number) immortalized B-cells (Eμ-Myc;Arf−/−) are not sensitive to AICAR, whereas aneuploid B cells (Eμ-Myc;p53−/−) cells are. Moreover, AICAR effect is indeed mediated by AMPK. Knock-down of AMPK allows Ep-Myc;p53−/− cells to proliferate the same in the presence and absence of AICAR (FIG. 2B).

FIG. 3A demonstrates that 17-AAG preferentially inhibited the proliferation of trisomic cells. Proliferation of trisomy 1, 13, 16 and 19 cells was impaired at a concentration of 100 nM of 17-AAG. Furthermore, cells trisomic for the largest chromosomes 1 exhibited higher sensitivity to the compound than cells harboring an additional copy of the smaller chromosomes 16 or 19.

FIG. 3B shows that chloroquine also preferentially inhibited the proliferation of aneuploid cells. As observed for AICAR and 17-AAG, the increased sensitivity of aneuploid cells correlated with the size of the additional chromosome.

The combined treatment of trisomic cells with AICAR and 17-AAG severely impaired their proliferative abilities but had little effect on euploid control cultures (FIG.

3C). Similar results were obtained when cells were treated with a combination of AICAR and chloroquine (FIG. 3D).

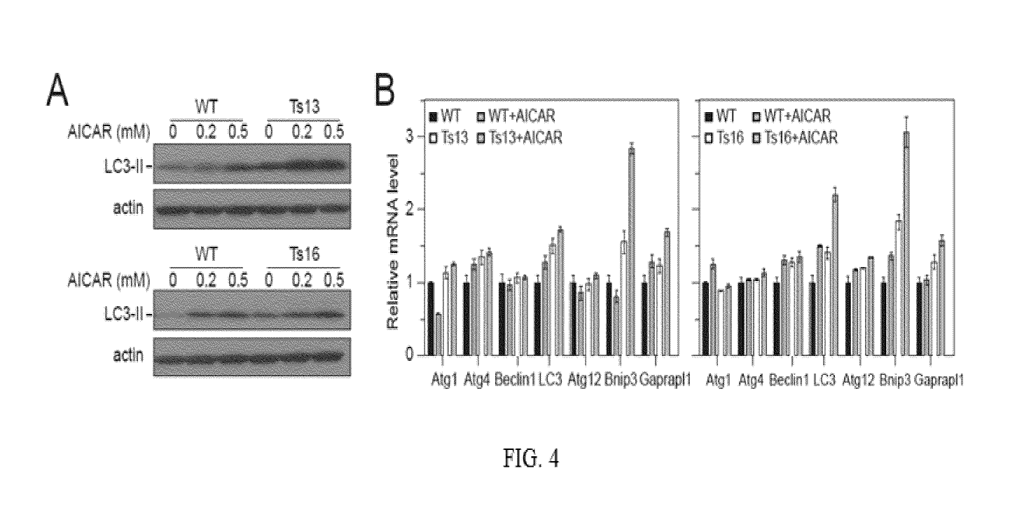

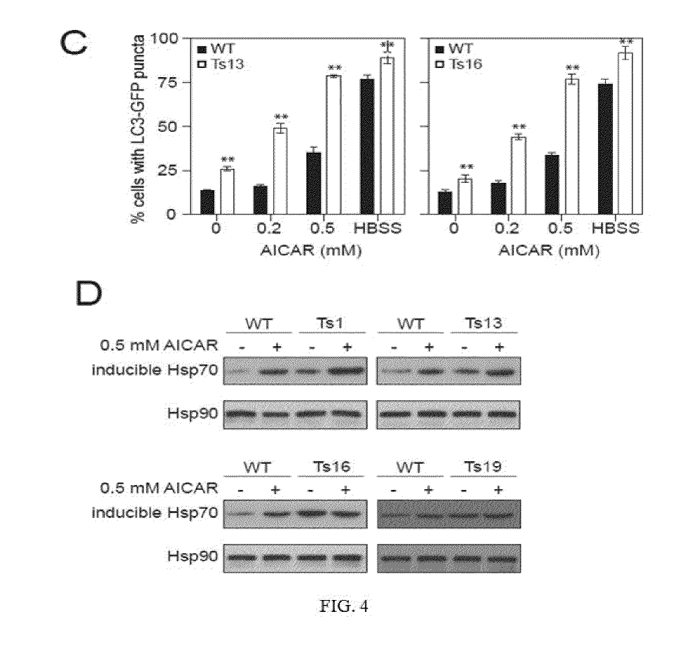

To determine whether trisomic MEFs are also under proteotoxic stress basal levels of autophagy and Hsp70 chaperone activity in trisomic MEFs and their behavior in response to AICAR treatment was examined. During autophagy, the autophagosomal membrane component LC3 is lipidated and incorporated into autophagosomal structures (Mizushima et al., 2008). In the absence of AICAR, trisomy 13 and 16 cells contained increased levels of LC3 mRNA and lipidated LC3 that was incorporated into autophagosomes (FIG. 4A-FIG. 4C). Expression of BNIP-3, a component of the autophagy machinery that is induced by many different stresses (Burton and Gibson, 2009; Mizushima and Klionsky, 2007), was also increased in trisomy 13 and 16 MEFs (FIG. 4B). AICAR treatment further induced Bnip3 expression as well as LC3 expression and its incorporation into autophagosomes (FIG. 4A-FIG. 4C).

Trisomic MEFs also harbor increased levels of the inducible form of the chaperone Hsp70 (FIG. 4D). AICAR treatment led to a further increase in Hsp70 activity (FIG. 4D). Thus, the activity of protein quality control pathways is elevated in aneuploid MEFs. The results further indicate that AICAR enhances the proteotoxic and energy stress present in aneuploid cells and that this enhancement contributes to the aneuploidy-selective anti-proliferative effects of AICAR.

### REFERENCES

Williams, B. R. et al. Aneuploidy affects proliferation and spontaneous immortalization in mammalian cells. Science. 322, 703-709 (2008).
Wendel, H. G. et al. Determinants of sensitivity and resistance to rapamycin-chemotherapy drug combinations in vivo. Cancer Res. 66, 7639-7646 (2006).
Silva, J. M. et al. Second-generation shRNA libraries covering the mouse and human genomes. Nat. Genet. 37, 1281-1288 (2005).
Huesken D, et al. Design of a genome-wide siRNA library using an artificial neural network. Nat. Biotechnol. 23, 995-1001 (2005).
Hemann, M. T. et al. An epi-allelic series of p53 hypomorphs created by stable RNAi produces distinct tumor phenotypes in vivo. Nat. Genet. 33, 396-400 (2003).
Dickins R A, et al. Probing tumor phenotypes using stable and regulated synthetic microRNA precursors. Nat. Genet. 37,1289-1295. (2005).
Young J C et al. Hsp90: a specialized but essential protein-folding tool. J Cell Biol 154, 267-73 (2001)
Glaumann H et al. Comparison of different autophagic vacuoles with regard to ultrastructure, enzymatic composition, and degradation capacity—formation of crinosomes.
Exp Mol Pathol 47, 346 (1987)
Levine B et al. Autophagy in the pathogenesis of disease. Cell 132, 27 (2008)
Mizushima et al. Autophagy fights disease through cellular self-digestion. Nature 451, 1069 (2008)
Burton, T. R., and Gibson, S. B. (2009). The role of Bcl-2 family member BNIP3 in cell death and disease: NIPping at the heels of cell death. Cell Death Differ 16, 515-523.
Mizushima, N., and Klionsky, D. J. (2007). Protein turnover via autophagy: implications for metabolism. Annu Rev Nutr 27, 19-40.
Torres, E. M., Sokolsky, T., Tucker, C. M., Chan, L. Y., Boselli, M., Dunham, M. J., and Amon, A. (2007). Effects of aneuploidy on cellular physiology and cell division in haploid yeast. Science 317, 916-924.

This invention is not limited in its application to the details of construction and the arrangement of components set forth in the above description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

We claim:

1. A method comprising,
contacting aneuploid cells with an AMP kinase agonist in an amount effective to reduce or inhibit the proliferation of the cells.

2. The method of claim 1, wherein said AMP kinase agonist is selected from the group consisting of Metformin (N,N-dimethylimidodicarbonimidic diamide), Resveratrol (trans-3,5,4′-Trihydroxystilbene), AICA-riboside (5-aminoimidazole-4-carboxamide riboside), AICA base (5-aminoimidazole-4-carboxamide), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues, and derivatives of any of said compounds.

3. The method of claim 1, further comprising contacting the cells with an effective amount of a therapeutic agent.

4. The method of claim 3, wherein the therapeutic agent is selected from the group consisting of: 17-(Allylamino)-17-demethoxygeldanamycin (17-AAG), chloroquine, 5-fluorouracil (5-FU), daunomycin, doxorubicin, mitomycin, paclitaxel, rapamycin, (sirolimus), dexamethasone, 2-methoxyestradiol, and Piritrexim isethionate.

5. A method comprising,
(a) identifying an individual on the basis that the individual is known to have aneuploid tumors;
(b) administering to the individual an AMP kinase agonist in an amount effective to reduce or inhibit the proliferation of aneuploid tumor cells in the tumor.

6. The method of claim 5, wherein the AMP kinase agonist is administered in combination with a therapeutic agent.

7. The method of claim 6, wherein the therapeutic agent can target proliferating tumor cells.

8. The method of claim 5, wherein said AMP kinase agonist is selected from the group consisting of Metformin (N,N-dimethylimidodicarbonimidic diamide), Resveratrol (trans-3,5,4′-Trihydroxystilbene), AICA-riboside (5-aminoimidazole-4-carboxamide riboside), AICA base (5-aminoimidazole-4-carboxamide), SAICAR (5-amino-4-imidazole-N-succinocarboxamide riboside), ZMP (5-aminoimidazole-4- carboxamide-1-beta-D-ribofuranosyl-5′-monophosphate), 6-MPR (6-mercaptopurine riboside), AMP analogues, and derivatives of any of said compounds.

9. The method of claim 7, wherein the therapeutic agent is selected from the group consisting of: 17-(Allylamino)-17-demethoxygeldanamycin (17-AAG), chloroquine, 5-fluorouracil (5-FU), daunomycin, doxorubicin, mitomycin, paclitaxel, rapamycin, (sirolimus), dexamethasone, 2-methoxyestradiol, and Piritrexim isethionate.

10. A method comprising:
(i) obtaining a tumor sample from an individual,
(ii) performing a karyotype analysis on the tumor sample to determine if tumor cells are aneuploid, wherein the presence of aneuploid cells indicates that an AMP kinase agonist should be administered to the individual.

11. The method of claim 10 further comprising administering to the individual an AMP kinase agonist in an amount effective to reduce or inhibit the proliferation of the tumor cells.

12. The method of claim 10, wherein said obtaining tumor sample involves a biopsy.

13. The method of claims 10, wherein said tumor sample is a cellular sample.

14. The method claim 10, wherein said tumor sample is a DNA sample.

15. The method of claim 11, wherein the AMP kinase agonist is administered in combination with a therapeutic agent.

16. The method of claim 15, wherein the therapeutic agent can target proliferating tumor cells.

17. The method of claim 10, wherein said AMP kinase agonist is selected from the group consisting of Metformin (N,N-dimethylimidodicarbonimidic diamide), Resveratrol (trans-3,5,4′-Trihydroxystilbene), AICA-riboside (5-aminoimidazole-4-carboxamide riboside), AICA base (5-aminoimidazole-4-carboxamide), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues, and derivatives of any of said compounds.

18. The method of claim 15, wherein the therapeutic agent is selected from the group consisting of: 17-(Allylamino)-17-demethoxygeldanamycin (17-AAG), chloroquine, 5-fluorouracil (5-FU), daunomycin, doxorubicin, mitomycin, paclitaxel, rapamycin, (sirolimus), dexamethasone, 2-methoxyestradiol, and Piritrexim isethionate.

19. A method comprising,
contacting tumor cells lacking functional endogeneous tumor suppressor gene with an AMP kinase agonist in an amount effective to reduce or inhibit the proliferation of the cells.

20. The method of claim 19, wherein the tumor suppressor gene encodes p53 protein.

21. The method of claim 19, wherein said AMP kinase agonist is selected from the group consisting of Metformin (N,N-dimethylimidodicarbonimidic diamide), Resveratrol (trans-3,5,4′-Trihydroxystilbene), AICA-riboside (5-aminoimidazole-4-carboxamide riboside), AICA base (5-aminoimidazole-4-carboxamide), SAICAR (5-amino-4-imidazole-N-succinocarboxamide riboside), ZMP (5-aminoimidazole-4-carboxamide-1-beta-D-ribofuranosyl-5′-monophosphate), 6-MPR (6-mercaptopurine riboside), AMP analogues, and derivatives of any of said compounds.

22. The method of claim 19, further comprising contacting the cells with an effective amount of a therapeutic agent.

23. The method of claim 22, wherein the therapeutic agent is selected from the group consisting of: 17-(Allylamino)-17-demethoxygeldanamycin (17-AAG), chloroquine, 5-fluorouracil (5-FU), daunomycin, doxorubicin, mitomycin, paclitaxel, rapamycin, (sirolimus), dexamethasone, 2-methoxyestradiol, and Piritrexim isethionate.

24. The method of claim 1, wherein the tumor is non-pancreatic.

25. The method of claim 1, wherein the AMP kinase agonist is not metformin.

26. The method of claim 1, wherein the AMP kinase agonist is not AICA-riboside.
